(19) United States
(12) Patent Application Publication
(10) Pub. No.: US 2024/0110913 A1
(43) Pub. Date: Apr. 4, 2024

(54) A MULTIPLEX ASSAY FOR THE DIAGNOSIS OF BRUCELLA CANIS INFECTION

(71) Applicant: CORNELL UNIVERSITY, Ithaca, NY (US)

(72) Inventors: Cassandra GUARINO, Ithaca, NY (US); Bettina WAGNER, Ithaca, NY (US); Craig ALTIER, Ithaca, NY (US)

(73) Assignee: CORNELL UNIVERSITY, Ithaca, NY (US)

(21) Appl. No.: 18/285,920

(22) PCT Filed: Apr. 7, 2022
(86) PCT No.: PCT/US2022/023811

§ 371 Date: Oct. 6, 2023

Related U.S. Application Data
(60) Provisional application No. 63/171,638, filed on Apr. 7, 2021.

(51) Int. Cl. G01N 33/569; G01N 33/58

(52) U.S. Cl. CPC G01N 33/56911; G01N 33/585

(57) ABSTRACT

A multiplex assay with an improved sensitivity and specificity for the diagnosis of Brucella canis infection in mammals using two specific antigens (BP26 and Omp31pep) is disclosed. Also disclosed are kits for detecting immune responses to a Brucella canis infection, methods of detection, methods for monitoring progression and methods for treating a Brucella canis infection.

### CROSS REFERENCE TO RELATED APPLICATION

This application claims the benefit of priority from U.S. Provisional Application No. 63/171,638, filed on Apr. 7, 2021, the contents of which are incorporated herein by reference.

### FIELD OF THE DISCLOSURE

The present disclosure describes an improved procedure to detect immune responses to pathogens (Brucella canis) using a multiplex assay system.

### INCORPORATION BY REFERENCE OF SEQUENCE LISTING

The Sequence Listing in an ASCII text file, named as 39359WO_9300_02_PC.SequenceListing of 10 KB, created on Apr. 5, 2022, and submitted to the United States Patent and Trademark Office via EFS-Web, is incorporated herein by reference.

### BACKGROUND

Efficient and accurate diagnosis of pathological agents (pathogens e.g., Brucella canis) is critical for clinical care, surveillance activities, outbreak control, pathogenesis, academic research, vaccine development, and clinical trials.

Brucellosis is a zoonotic bacterial disease caused by several species in the genus Brucella. Brucella canis is an important cause of reproductive failure in dogs, especially in kennels. Infections can result in abortions and stillbirths in bitches, and epididymitis, prostatitis, orchitis and sperm abnormalities in males. Although spayed or neutered dogs do not have reproductive signs, they occasionally develop other conditions such as ocular disease and discospondylitis. B. canis may persist in an animal even after antibiotic treatment. In kennels, infected dogs are often euthanized to prevent them from infecting other dogs or people. Canine brucellosis is sometimes difficult to diagnose with the currently available tests. The importance of B. canis as a cause of human illness is still unclear. Few clinical cases have been reported in people, and most have been mild. However, human infections with this organism may be underdiagnosed, as the symptoms are nonspecific, diagnostic suspicion among physicians is low, and obtaining a definitive diagnosis may be difficult.

Traditional serologic assays for detection of Brucella canis infection include the rapid slide agglutination tests (RSAT), tube agglutination tests (TAT), and immunofluorescent antibody tests (IFA), which can be used for initial screening. As with any serologic assay, false negative reactions can occur in the early stages of infection, prior to seroconversion, and low circulating antibody titers can occur in some chronically infected dogs (Greene C E, Carmichael L E. Canine brucellosis. In: Infectious diseases of the dog and cat, 4th ed., 2012:398-411; Wanke M M. Canine brucellosis. Animal Reproduction Science 2004; 82-83:195-207; Wooley R E, et al. Isolation of Brucella canis from a dog seronegative for brucellosis. J Am Vet Med Assoc 1978; 173: 387-388).

The predominant disadvantage of these screening assays is the potential for cross-reactivity, both specific and non-specific, from shared surface antigens of other bacteria, leading to false positive reactions (Hollett R B. Canine brucellosis: Outbreaks and compliance. Theriogenology 2006; 66: 575-587). Therefore, these screening tests need to be followed by a reference assay, including slide agglutination (2ME-RSAT) and agar gel immunodiffusion (AGID), for confirmation.

To our knowledge, there does not exist a current technology that can overcome both the cross-reactivity concerns of the B. canis screening tests and reagent production difficulty of the reference assay while maintaining high sensitivity and specificity for B. canis diagnosis.

### SUMMARY OF THE DISCLOSURE

An aspect of the disclosure is directed to a kit for detecting immune responses to a Brucella canis infection in a mammal comprising:
a. a multiplex capture reagent comprising a first antigenic peptide from a Brucella canis BP26 protein linked to a first detectable label, and a second antigenic peptide from a Brucella canis Omp31 protein linked to a second detectable label;
b. a detection antibody against antibodies of the mammal, wherein the detection antibody is linked to a third detectable label, and wherein the first detectable label, the second detectable label and the third detectable labels are different from each other;
c. and optionally a solid substrate and instructions for detecting the presence of antibodies to Brucella canis.

In some embodiments, the BP26 protein comprises the amino acid sequence of SEQ ID NO: 1 or an amino acid sequence at least 95% identical to SEQ ID NO: 1.

In some embodiments, the first antigenic peptide comprises at least 10, at least 20, at least 30, at least 40, at least 50, at least 60, at least 70, at least 80, at least 90, at least 100 amino acids, at least 120 amino acids, at least 150 amino acids, at least 180 amino acids, at least 200 amino acids, at least 220 amino acids or all of the BP26 protein.

In some embodiments, the first antigenic peptide comprises a fragment of the BP26 protein without the N-terminal transmembrane domain of the BP26 protein.

In some embodiments, the Omp31 protein comprises the amino acid sequence of SEQ ID NO: 2 or an amino acid sequence at least 95% identical to SEQ ID NO: 2. In another embodiment, the second antigenic peptide comprises at least 10 amino acids of the Omp31 protein.

In some embodiments, the second antigenic peptide comprises an extracellular region of the Omp31 protein. In another embodiment, the extracellular region of the Omp31 protein comprises an amino acid sequence that is at least 95% identical to SEQ ID NO: 8. In another embodiment, the extracellular region of the Omp31 protein comprises an amino acid sequence as shown in SEQ ID NO: 8 or 9.

In some embodiments, the first detectable label is a first fluorescent bead and the second detectable label is a second fluorescent bead. In another embodiment, the first detectable label, the second detectable label and the third detectable label provide fluorescent signals.

In some embodiments, the mammal is a dog or a human.

In some embodiments, the solid support is selected from a multi-well plate or a laminar flow device.

In some embodiments, the capture reagent comprises a third antigenic peptide from a Brucella canis protein.

In another aspect, the disclosure is directed to a method for simultaneous detection of antibodies directed to multiple Brucella canis antigens in a mammal comprising:
(a) providing a capture reagent comprising a first antigenic peptide from a BP26 protein of Brucella canis linked to a first detectable label, and a second antigenic peptide from an Omp31 protein of Brucella canis linked to a second detectable label, wherein the first detectable label and the second detectable label are different from each other;
(b) contacting the capture reagent with a biological sample from the mammal to allow for antibodies present in the sample to bind to the antigenic peptides in the capture reagent, thereby forming a first complex between the first antigenic peptide and an antibody, and a second complex between the second antigenic peptide and an antibody;
(c) contacting the capture reagent after step (b) with a detection antibody, wherein the detection antibody is linked to a third detectable label that is different from the first detectable label and the second detectable label, to allow for the detection antibody to bind to the antibodies in the first and second complexes; and
(d) detecting the detection antibody bound to the antibody in first complex and detecting detection antibody bound to the antibody in the second complex.

In some embodiments, the method for simultaneous detection of antibodies further comprises quantifying the amount of detection antibody bound to the antibody in the first complex and the amount of detection antibody bound to the antibody in the second complex.

In some embodiments, the BP26 protein comprises the amino acid sequence of SEQ ID NO: 1 or an amino acid sequence at least 95% identical to SEQ ID NO: 1.

In some embodiments, the first antigenic peptide comprises at least 10, at least 20, at least 30, at least 40, at least 50, at least 60, at least 70, at least 80, at least 90, at least 100 amino acids, at least 120 amino acids, at least 150 amino acids, at least 180 amino acids, at least 200 amino acids, at least 220 amino acids or all of the BP26 protein.

In some embodiments, the first antigenic peptide comprises a fragment of the BP26 protein without the N-terminal transmembrane domain of the BP26 protein. In some embodiments, the first antigenic peptide comprises a fragment of a BP26 protein which corresponds to amino acid residues from 28 to 250 of a BP26 protein, e.g., amino acids 28-250 of the BP protein as shown in SEQ ID NO: 1.

In some embodiments, the Omp31 protein comprises the amino acid sequence of SEQ ID NO: 2 or an amino acid sequence at least 95% identical to SEQ ID NO: 2. In another embodiment, the second antigenic peptide comprises at least 10 amino acids of the Omp31 protein.

In some embodiments, the second antigenic peptide comprises an extracellular region of the Omp31 protein. In another embodiment, the extracellular region of the Omp31 protein comprises an amino acid sequence that is at least 95% identical to SEQ ID NO:8. In another embodiment, the extracellular region of the Omp31 protein comprises an amino acid sequence as shown in SEQ ID NO: 8 or 9.

In some embodiments, the first detectable label is a first fluorescent bead and the second detectable label is a second fluorescent bead. In another embodiment, the first detectable label, the second detectable label and the third detectable label are fluorescent labels. In another embodiment, the detection is achieved by a flow cytometer or a plate reader.

In some embodiments, the mammal is a dog or a human.

In some embodiments, the biological sample is selected from blood, plasma, serum, saliva, urine, synovial and cerebrospinal fluid, milk and tissue homogenates.

In some embodiments, a combination of a positive detection of the detection antibody bound to the antibody in first complex and a positive detection of the detection antibody bound to the antibody in the second complex is indicative of a Brucella canis infection.

In a further aspect, the disclosure is directed to a method for monitoring progression of a Brucella canis infection in a mammal comprising:
(a) providing a plurality of biological samples collected from the mammal, wherein each biological sample is collected from the mammal at a different time point;
(b) measuring the amount of antibodies directed to multiple Brucella canis antigens in each biological sample,
   wherein each measuring comprises:
(i) providing a biological sample from one time point;
(ii) providing a capture reagent comprising a first antigenic peptide from a BP26 protein of Brucella canis linked to a first detectable label, and a second antigenic peptide from an Omp31 protein of Brucella canis linked to a second detectable label, wherein the first detectable label and the second detectable label are different from each other;
(iii) contacting the capture reagent with the biological sample to allow for antibodies present in the sample to bind to the antigenic peptides in the capture reagent, thereby forming a first complex between the first antigenic peptide and an antibody, and a second complex between the second antigenic peptide and an antibody;
(iv) contacting the capture reagent after step (iii) with a detection antibody, wherein the detection antibody is linked to a third detectable label that is different from the first detectable label and the second detectable label, to allow for the detection antibody to bind to the antibodies in the first and second complexes,
(v) quantifying the amount of detection antibody bound to the antibody in the first complex and the amount of detection antibody bound to the antibody in the second complex; and
(vi) comparing the amounts measured in step (b) for the different time points, thereby monitoring the progression of Brucella canis infection.

In some embodiments, the different time points comprise every 6 hours, every 12 hours, every day, every 2 days, every 3 days, every 4 days, every 5 days, every 6 days, every 7 days, every 8 days, every 9 days, every 10 days, every two weeks or every three weeks. In another embodiment, the different time points comprise every week, every month, every 2 months, every 3 months, every 4 months, every 6 months, and/or every year.

In some embodiments, the BP26 protein comprises the amino acid sequence of SEQ ID NO: 1 or an amino acid sequence at least 95% identical to SEQ ID NO: 1. In another embodiment, the first antigenic peptide comprises at least 10, at least 20, at least 30, at least 40, at least 50, at least 60, at least 70, at least 80, at least 90, at least 100 amino acids, at least 120 amino acids, at least 150 amino acids, at least 180 amino acids, at least 200 amino acids, at least 220 amino acids or all of the BP26 protein.

In some embodiments, the first antigenic peptide comprises a fragment of the BP26 protein without the N-terminal transmembrane domain of the BP26 protein.

In some embodiments, the Omp31 protein comprises the amino acid sequence of SEQ ID NO: 2 or an amino acid sequence at least 95% identical to SEQ ID NO: 2. In another embodiment, the second antigenic peptide comprises at least 10 amino acids of the Omp31 protein.

In some embodiments, the second antigenic peptide comprises an extracellular region of the Omp31 protein. In another embodiment, the extracellular region of the Omp31 protein comprises an amino acid sequence that is at least 95% identical to SEQ ID NO:8. In another embodiment, the extracellular region of the Omp31 protein comprises an amino acid sequence as shown in SEQ ID NO: 8 or 9.

In some embodiments, the first detectable label is a first fluorescent bead and the second detectable label is a second fluorescent bead. In another embodiment, the first detectable label, the second detectable label and the third detectable label are fluorescent labels. In another embodiment, the detection is achieved by a flow cytometer or a plate reader.

In some embodiments, the mammal is a dog or a human.

In some embodiments, the biological sample is selected from blood, plasma, serum, saliva, urine, synovial and cerebrospinal fluid, milk and tissue homogenates.

In another aspect, the disclosure is directed to a method for treating Brucella canis in a mammal comprising:
(a) obtaining a sample from a mammal;
(b) detecting antibodies reactive to a first antigenic peptide from a BP26 protein of Brucella canis in the sample;
(c) detecting antibodies reactive to a second antigenic peptide from an Omp31 protein of Brucella canis in the sample;
(d) treating the mammal based on the detection of the first antigenic peptide and/or second antigenic peptide.

In some embodiments, the method further comprises quantifying an amount of antibodies to the first antigenic peptide and/or quantifying an amount of antibodies to the second antigenic peptide.

In some embodiments, the BP26 protein comprises the amino acid sequence of SEQ ID NO: 1 or an amino acid sequence at least 95% identical to SEQ ID NO: 1. In another embodiment, the first antigenic peptide comprises at least 10, at least 20, at least 30, at least 40, at least 50, at least 60, at least 70, at least 80, at least 90, at least 100 amino acids, at least 120 amino acids, at least 150 amino acids, at least 180 amino acids, at least 200 amino acids, at least 220 amino acids or all of the BP26 protein.

In some embodiments, the first antigenic peptide comprises a fragment of the BP26 protein without the N-terminal transmembrane domain of the BP26 protein.

In some embodiments, the Omp31 protein comprises the amino acid sequence of SEQ ID NO: 2 or an amino acid sequence at least 95% identical to SEQ ID NO: 2. In another embodiment, the second antigenic peptide comprises at least 10 amino acids of the Omp31 protein.

In some embodiments, the second antigenic peptide comprises an extracellular region of the Omp31 protein. In another embodiment, the extracellular region of the Omp31 protein comprises an amino acid sequence that is at least 95% identical to SEQ ID NO: 8. In another embodiment, the extracellular region of the Omp31 protein comprises an amino acid sequence as shown in SEQ ID NO: 8 or 9.

In some embodiments, detecting antibodies reactive to a first antigenic peptide from a BP26 protein of Brucella canis in the sample and/or detecting antibodies reactive to a second antigenic peptide from an Omp31 protein of Brucella canis in the sample comprises an ELISA assay, a fluorescence assay, and/or a colorimetric assay.

In some embodiments, the mammal is a dog or a human.

In some embodiments, the biological sample is selected from blood, plasma, serum, saliva, urine, synovial and cerebrospinal fluid, milk and tissue homogenates.

In some embodiments, a combination of a positive detection of antibodies reactive to a first antigenic peptide from a BP26 protein of Brucella canis in the sample and a positive detection of antibodies reactive to a second antigenic peptide from an Omp31 protein of Brucella canis is indicative of a B. canis infection in the mammal.

In some embodiments, treating comprises treating with an antibiotic, treating with more than one antibiotic, treating with an aminoglycoside, monitoring disease state, and/or quarantining the mammal.

### BRIEF DESCRIPTION OF THE DRAWINGS

FIG. 1A: Illustration of the steps of the multiplex assay for detection of antibodies to Brucella canis. (1) B. canis antigens, BP26 and Omp31pep (or PO1), are coupled to fluorescent beads. (2) Canine serum samples are incubated with the beads. (3) A biotinylated anti-canine immunoglobulin antibody is added. (4) Streptavidin-phycoerythrin (PE) is added as a reporter.

The bead and associated PE fluorescence is measured.

FIG. 1B: BP26 Protein and Omp31 peptide sequences. His tagged BP26 Protein Sequence (SEQ ID NO: 7), Omp31pep (fragment of Omp31protein) Sequence (SEQ ID NO: 8) and PO1 (fragment of Omp31pep) Sequence (SEQ ID NO: 9).

FIGS. 2A-B: Result distribution of samples submitted to the AHDC (Cornell University, Ithaca, NY) for B. canis. Samples were considered Negative (A. n=1784, B. n=747) if serum was not reactive on both AGIDII and Slide Agglutination. Samples were considered Inconclusive (A. n=60, B. n=33) if negative on AGID II but positive on Slide agglutination or suspect on AGIDII regardless of Slide Agglutination result. Samples were considered Positive (A. n=130, B. n=87) if positive on the AGID II assay, regardless of Slide Agglutination result. BP26, Omp31pep, and PO1 MFI results can be used independently to distinguish positive and negative results (Mann Whitney test p<0.0001). Dotted lines represent cut-off values for optimized sensitivity and specificity derived from the ROC curves, as compared to the reference assay.

FIGS. 3A-B: Linear combination of multiplex results. A linear regression model combining the BP26 and A. Omp31pep (n=1784) or B. PO1 (n=747) MFI results demonstrates the improved separation of positive and negative samples. For this combined analysis, box and whiskers plot of the probability of a sample being positive are represented—values outside of the 1-99 percentile are denoted as points on the graph. Dotted line represents the probability value at which the sensitivity and specificity are optimized, based on ROC analysis.

FIGS. 4A-B: ROC curves. Utilizing the data represented in FIG. 3, with the ‘Inconclusive’ samples removed (A. n=60, B. n=33), receiver operating characteristic curves were produced for each antigen alone, and in combination. A. Area under the curve (AUC) for BP26 is 0.930, for Omp31pep is 0.907, and for the linear combination the two antigens, AUC=0.957. B. Area under the curve for BP26 is 0.911, for PO1 is 0.914, and for the linear combination of the two antigens, AUC=0.972.

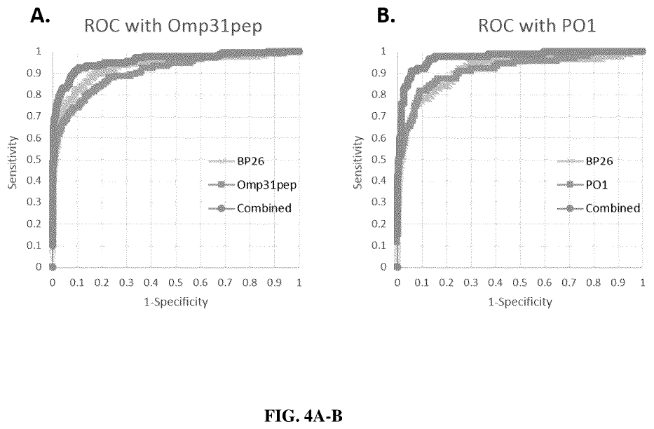

FIG. 5: Peptide analysis of Omp31. Peptides PO1-PO5 cover the Omp31pep with overlap, including flanking regions. Peptides UO2-UO5 cover other regions of the Omp31 protein. Each peptide, conjugated to BSA and coupled to beads, was probed for reactivity with total Ig from canine serum, including serum from known B. canis negative animals (‘Neg’, n=7), and B. canis positive animals, as identified by the reference assay (‘Pos’, n=8). Only peptide PO1 was able to distinguish positive and negative animals. Peptide UO3 was extended to ‘UO3e’ by the addition of a ‘-GKKK’ linker prior to the cysteine residue at the C-terminus to improve solubility.

### DETAILED DESCRIPTION

The term “detecting” is used in the broadest sense to include both qualitative and quantitative measurements of a target molecule, such as an antibody in a biological sample (e.g., a serum sample).

The term “biological sample” includes body samples from an animal, including biological fluids such as serum, plasma, vitreous fluid, lymph fluid, synovial fluid, follicular fluid, seminal fluid, amniotic fluid, milk, whole blood, urine, cerebro-spinal fluid, saliva, sputum, tears, perspiration, mucus, and tissue culture medium, as well as tissue extracts such as homogenized tissue, and cellular extracts. In some embodiments, the biological sample is a serum sample.

The term “animal” includes mammals, for example, human, horse, camel, dog, pig, cow, and sheep. In some embodiments, the animal is a dog. In some embodiments, the animal is a human. In some embodiments, the animal is an animal suspected to have contracted a disease (e.g., an infection with a pathogen). In some embodiments, the animal is a dog or human suspected of suffering from a Brucella canis infection.

The term “antibody” is used in the broadest sense and includes monoclonal antibodies (including agonist, antagonist, and neutralizing antibodies), polyclonal antibodies, multivalent antibodies, multispecific antibodies, and antibody fragments so long as they exhibit the desired binding specificity.

The term “antigenic peptides” generally refer to peptides capable of provoking an immune response in a host animal. Antigenic peptides are of sufficient length to be antigenic, i.e., provoking an immune response in a host animal. In some embodiments, antigenic peptides are at least 12-15 amino acids in length. In some embodiments, antigenic peptides are at least 20 amino acids, at least 25 amino acids, at least 30 amino acids, at least 35 amino acids, at least 40 amino acids in length, or at least 50 amino acids. In some embodiments, antigenic peptides can be up to 250 amino acids or more. In some embodiments, antigenic peptides comprise not more than 250 amino acids, not more than 200 amino acids, not more than 150 amino acids, not more than 100 amino acids, or not more than 75 amino acids in length. In some embodiments, antigenic peptides are peptide fragments of a native target protein.

The term “capture reagent” generally refers to a reagent capable of binding and capturing a target molecule in a sample.

The term “plasmids” includes both naturally occurring plasmids in bacteria, and artificially constructed circular DNA fragments.

The term “expression vector” refers to a nucleic acid that includes sequences that effect the expression of a desirable molecule, e.g., a promoter, a coding region and a transcriptional termination sequence. An expression vector can be an integrative vector (i.e., a vector that can integrate into the host genome), or a vector that does not integrate but self-replicates, in which case, the vector includes “ ” an origin of replication which permits the entire vector to be reproduced once it is within the host cell.

The term “cloning site” refers to a nucleotide sequence, typically present in an expression vector, that includes one or more restriction enzyme recognition sequences useful for cloning a DNA fragment(s) into the expression vector.

This disclosure provides a novel diagnostic assay for the confirmation of Brucella canis infection in dogs. This assay is based on simultaneous detection of antibodies against multiple Brucella canis antigens, for example, a BP26 protein or a peptide fragment thereof, and a peptide derived from the outer membrane protein, Omp31. The disclosure provides a clear improvement compared to already available assays for Brucella canis. Specially, the novel Brucella canis assay offers high sensitivity and specificity for B. canis diagnosis; overcomes the cross-reactivity concerns of the currently available Brucella canis screening tests and avoids difficulties of preparation of reagents required for production of the reference assay. The current reference assay for diagnosis of B. canis requires the regular growth of this biosafety level 3 (BSL3) zoonotic pathogen. In addition, the current reference assay requires control antigen from animals with known history of B. canis infection.

This novel assay has the potential to be simultaneously used as both a screening tool and a confirmatory assay instead of running the 2ME-RSAT and AGID assays described above. Sensitivity of the novel assay is improved compared to the existing reference assays, while specificity is improved over the screening tests. The novel assay can precisely quantify the amount of each of multiple Brucella antigen-specific antibodies present in an animal serum sample. The quantitative value can be monitored over time to verify both exposure status and may prove useful for monitoring the effectiveness of treatment.

In some embodiments, a kit for detecting immune responses to a Brucella canis infection in a mammal is provided. Brucellosis is a zoonotic bacterial disease caused by several species in the genus Brucella, which mainly infect cattle, swine, goats, sheep and dogs. Humans generally acquire the disease through direct contact with infected animals, by eating or drinking contaminated animal products or by inhaling airborne agents. In dogs, brucellosis is mainly caused by Brucella canis, a Gram-negative coccobacillus in the family Brucellaceae (class Alphaproteobacteria). It can also cause infection in humans. In some embodiments, a mammal is a dog or human.

In some embodiments, the kit provides components for a multiplex assay system which detects antibodies to several Brucella canis antigens simultaneously. To perform the present assays for detecting immune responses to an antigenic entity (e.g., a pathogen), the kit includes a capture reagent, a detection antibody, and optionally a solid substrate and instructions for detecting the presence of antibodies to Brucella canis.

A capture reagent included in a kit serves to capture antibodies in a biological sample that are directed to antigens of Brucella canis. The capture reagent disclosed herein is also referred to as a multiplex capture reagent because it includes multiple (i.e., two or more) antigenic peptides of Brucella canis proteins. In some embodiments, a capture reagent comprises a first antigenic peptide from a Brucella canis BP26 protein and a second antigenic peptide from a Brucella canis Omp31 protein.

In some embodiments, the first antigenic peptide from the Brucella canis BP26 protein is linked to a first detectable label and the second antigenic peptide from the Brucella canis Omp31 protein is linked to a second detectable label, wherein the first detectable label and the second detectable label are different from each other. In some embodiments, detectable labels are fluorescent beads which provide fluorescent signals. Hence, the first antigenic peptide from the Brucella canis BP26 protein can be linked to a first fluorescent bead and the second antigenic peptide from the Brucella canis Omp31 protein is linked to a second fluorescent bead, wherein the first and second beads provide different fluorescent signals.

An exemplary full-length Brucella canis BP26 (GenBank ID: AIJ81780.1) protein sequence (SEQ ID NO: 1) is shown below:

MNTRASNFLAASFSTIMLVGAFSLPAFAQENQMTTQPARI
AVTGEGMMTASPDMAILNLSVLRQAKTAREAMTANNEAMT
KVLDAMKKAGIEDRDLQTGGINIQPIYVYPDDKNNLKEPT
ITGYSVSTSLTVRVRELANVGKILDESVTLGVNQGGDLNL
VNDNPSAVINEARKRAVANAIAKAKTLADAAGVGLGRVVE
ISELSRPPMPMPIARGQFRTMLAAAPDNSVPIAAGENSYN
VSVNVVFEIK*

In some embodiments, a BP26 protein comprises an amino acid sequence that is substantially identical (e.g., at least 95%, at least 98% or at least 99% identical) with SEQ ID NO: 1. The variations from SEQ ID NO: 1 can be natural occurring, e.g., as a result of polymorphism.

In some embodiments, the first antigenic peptide comprises at least 10, at least 20, at least 30, at least 40, at least 50, at least 60, at least 70, at least 80, at least 90, at least 100 amino acids, at least 120 amino acids, at least 150 amino acids, at least 180 amino acids, at least 200 amino acids, at least 220 amino acids or all of a BP26 protein, such as the BP26 protein as shown in SEQ ID NO: 1 or a BP26 protein substantially identical with SEQ ID NO: 1. Alternatively, the first antigenic peptide comprises at least 10%, 20%, 30%, 40%, 50%, 60%, 70%, 80%, 90%, 95%, 99% or more of the BP26 protein as shown in SEQ ID NO: 1. In some embodiments, the first antigenic peptide comprises not more than 220 amino acids, not more than 200 amino acids, not more than 180 amino acids, or not more than 150 amino acids of a BP26 protein, such as the BP26 protein as shown in SEQ ID NO: 1 or a BP26 protein substantially identical with SEQ ID NO: 1. In some embodiments, the first antigenic peptide comprises about 50 amino acids to about 250 amino acids. In some embodiments, the first antigenic peptide comprises about 75 amino acids to about 220 amino acids. In some embodiments, the first antigenic peptide comprises about 100 amino acids to about 200 amino acids. In some embodiments, the first antigenic peptide comprises a fragment of a BP26 protein that is devoid of the N-terminal transmembrane domain. The N-terminal transmembrane domain of a BP26 protein spans from amino acid residues 7 to 29, e.g., amino acids 7-29 of SEQ ID NO: 1. In some embodiments, the first antigenic peptide comprises a fragment of a BP26 protein which corresponds to amino acid residues from 28 to 250 of a BP26 protein, e.g., amino acids 28-250 of the BP protein as shown in SEQ ID NO: 1. In some embodiments, the first antigenic peptide comprising amino acid residues from 28 to 250 of a BP26 protein is expressed as a cytoplasmic protein (i.e., without a signal sequence) in E. coli.

An exemplary full-length Brucella canis Omp31 (Genbank ID: AAL27296.1) protein sequence (SEQ ID NO: 2) is shown below:

MKSVILASIAAMFATSAMAADVVVSEPSAPTAAPVDTFSW
TGGYIGINAGYAGGKFKHPFSSFDKEDNEQVSGSLDVTAG
GFVGGVQAGYNWQLDNGVVLGAETDFQGSSVTGPISAGAS
GLEGKAETKVEWFGTVRARLGYTATERLMVYGTGGLAYGK
VKSAFNLGDDASALHTWSDKTKAGWTLGAGAEYAINNNWT
LKSEYLYTDLGKRNLVDVDNSFLESKVNFHTVRVGLNYKF

In some embodiments, an Omp31 protein comprises an amino acid sequence that is substantially identical (e.g., at least 95%, at least 98% or at least 99% identical) with SEQ ID NO: 2. The variations from SEQ ID NO: 1 can be natural occurring, e.g., as a result of polymorphism.

In some embodiments, the second antigenic peptide comprises at least 10%, 20%, 30%, 40%, 50%, 60%, 70%, 80%, 90%, 95%, 99% or more of an Omp31 protein, such as the Omp31 protein as shown in SEQ ID NO: 2 or an Omp31 protein substantially identical to SEQ ID NO: 2.

In some embodiments, the second antigenic peptide comprises at least 10 amino acids of an Omp31 protein, such as the Omp31 protein as shown in SEQ ID NO: 2 or an Omp31 protein substantially identical to SEQ ID NO: 2.

In some embodiments, the second antigenic peptide comprises an extracellular region of an Omp31 protein, or an amino acid substantially identical (e.g., at least 95%, at least 98% or at least 99% identical) to an extracellular region of an Omp31 protein. In some embodiments, an extracellular region of an Omp31 protein comprises an amino acid sequence that is identical or substantially identical (e.g., at least 95%, at least 98% or at least 99% identical) to SEQ ID NO: 8 (Omp31pep). In some embodiments, the second antigenic peptide comprises the amino acid sequence as shown in SEQ ID NO: 8. The Omp31pep peptide represents amino acids 48-77 of the Omp31 protein shown in SEQ ID NO: 2, a region at the C-terminal domain of the Omp31 protein. A smaller 17aa peptide within the Omp31pep sequence was identified as PO1 Sequence (SEQ ID NO: 9) that has a further improved sensitivity and specificity. In some embodiments, the second antigenic peptide comprises the amino acid sequence as shown in SEQ ID NO: 9.

Additional peptide sequences are shown below. Peptides PO1-PO5 cover the Omp31pep with overlap, including flanking regions. Peptides UO2-UO5 cover other regions of the Omp31 protein. Peptide PO1 (SEQ ID NO: 9) is able to distinguish positive and negative animals.

PO1:
(SEQ ID NO: 9)
[NH2]GKFKHPFSSFDKEDNEQ[COOH]
PO2:
(SEQ ID NO: 10)
[NH2]NAGYAGGKFKHPFSS[COOH]
PO3:
(SEQ ID NO: 11)
[NH2]FDKEDNEQVSGSLDV[COOH]
PO4:
(SEQ ID NO: 12)
[NH2]GGYIGINAGYAGGKF[COOH]
PO5:
(SEQ ID NO: 13)
[NH2]EQVSGSLDVTAGGFVGGVQA[COOH]
UO2:
(SEQ ID NO: 14)
[NH2]MAADVVVSEPSAPTAAPVDT[COOH]
UO3:
(SEQ ID NO: 15)
[NH2]YNWQLDNGVVLGAETDFQGSSVTG*[COOH]
UO4:
(SEQ ID NO: 16)
[NH2]MVYGTGGLAYGKVKSAFNLG[COOH]
UO5:
(SEQ ID NO: 17)
[NH2]NSFLESKVNFHTVRVGLNYK[COOH]

Each peptide (SEQ ID NO: 9-17) can have an additional cysteine residue added to the C-terminus for conjugating to a carrier protein such as a maleimide-activated BSA carrier protein. The “*” after the UO3 sequence (SEQ ID NO: 15) is representative of the addition of a “GKKK” linker prior to the cysteine residue at the C-terminus to improve solubility.

In some embodiments, the antigenic peptides include an additional cysteine residue added to the C-terminus for conjugating to a carrier protein, e.g., a maleimide-activated BSA carrier protein. A carrier protein can facilitate conjugating an antigenic peptide to a solid material, e.g., beads.

In some embodiments, the capture reagent comprises a third antigenic peptide from a Brucella canis protein, which differs from the first and second antigenic peptides and can be a peptide of a BP26 protein, an Omp31 protein, or another Brucella canis protein (such as ABCt (WP_004687190.1), or HRL18 (NC_010103.1, c1079819-1079544)).

In addition to a capture reagent which binds to (“capture”) specific antibodies present in a biological sample of a mammal, a kit provided herein also include a detection antibody. A detection antibody, which is linked to a label, recognizes antibodies in the biological sample of a mammal and forms a complex with the antibodies. The complex formed can then be detected through the label linked to the detection antibody. In some embodiments, a detection antibody is an anti-canine immunoglobulin antibody, which is useful in instances where canine samples (such as serum samples) are used as biological samples in the multiplex assay. In some embodiments, a detection antibody is linked to biotin, which binds to streptavidin conjugated with a compound that provides a detectable signal (e.g., phycoerythrin). The first detectable label (linked to the first antigenic peptide), the second detectable label (linked to the second antigenic peptide), and the label (linked to a detection antibody) are different from each other.

In some embodiments, a kit includes a solid substrate. In some embodiments, the solid substrate is selected from a multiwell plate and a laminar flow device.

Further disclosed herein is a method for simultaneous detection of antibodies in a biological sample wherein the antibodies are directed to multiple Brucella canis antigens.

The present method includes
(a) providing a capture reagent described herein comprising multiple antigenic peptides of Brucella canis, each linked to a detectable label (for example, a capture reagent comprising a first antigenic peptide from a BP26 protein of Brucella canis linked to a first detectable label, and a second antigenic peptide from an Omp31 protein of Brucella canis linked to a second detectable label, wherein the first detectable label and the second detectable label are different from each other);
(b) contacting the capture reagent with a biological sample from the mammal to allow for antibodies present in the sample to bind to the multiple antigenic peptides in the capture reagent, thereby forming multiple complexes (for example, a first complex between the first antigenic peptide and an antibody, and a second complex between the second antigenic peptide and an antibody);
(c) contacting the capture reagent after step (b) with a detection antibody, to allow for the detection antibody to bind to the antibodies captured by the capture reagent in each of the multiple complexes (e.g., antibodies in the first and second complexes); and
(d) detecting the detection antibody bound to each of the multiple complexes.

In some embodiments, a method for simultaneous detection of antibodies directed to multiple Brucella canis antigens is disclosed in which each antigen is independently coupled to a unique fluorescent bead, as described above. The bead-based assay allows for a wide dynamic range for quantification of antibody present in a given sample. Two or more antigenic peptides are included in a capture reagent for optimal sensitivity and specificity. In some embodiments, one antigenic peptide is derived from a BP26 protein and another antigenic peptide is derived from an Omp31 protein (e.g., a PO1 peptide disclosed herein which provides more optimal sensitivity and specificity than the longer peptide, Omp31pep).

In some embodiments, the biological sample is selected from blood, plasma, serum, saliva, urine, synovial and cerebrospinal fluid, milk and tissue homogenates.

In some embodiments, a biological sample is added into the wells of a multi-well plate, followed by addition of a capture reagent (which includes antigenic peptides coupled to different fluorescent beads), thereby contacting the capture reagent with the biological sample from the mammal to allow for antibodies present in the sample to bind to the antigenic peptides in the capture reagent to form a first complex between the first antigenic peptide and an antibody, and a second complex between the second antigenic peptide and an antibody. Thereafter, a suitable wash buffer, e.g., phosphate buffered saline containing 0.05% Tween 20 (PBST), can be applied to the capture agent (e.g., peptide coupled fluorescent beads after incubated with a serum sample) and/or the multi-well plate to remove unbound antibodies in the biological sample. Next, a detection antibody (e.g., anti-canine immunoglobulin antibody) is added into the wells which binds to the antibodies in the first and second complexes if formed. The detection antibody is linked to a third detectable label, such as biotin, which can be detected by streptavidin conjugated with a compound that provides a detectable signal (e.g., phycoerythrin). The detectable signal is measured, and the data are represented as median fluorescent intensities (MFI). In some embodiments, the detection is measured by a flow cytometer or a plate reader.

In some embodiments, the amount of detection antibody bound to the antibody in the first complex and the amount of detection antibody bound to the antibody in the second complex are quantified. A combination of a positive detection of a detection antibody bound to the antibody in first complex and the positive detection antibody bound to the antibody in the second complex is indicative of a Brucella canis infection.

In some embodiments, the assay could be implemented in a laminar-flow type device or as a set of ELISAs. Alternatively, the antigens could be coated in a multiplex fashion as individual spots in an ELISA type assay.

In some embodiments, additional antigens (third, or fourth, or more antigenic peptides) can be added to the assay.

In some embodiments, a method for monitoring the progression of a Brucella canis infection in a mammal is disclosed. To monitor the progression of a Brucella canis infection, a plurality of biological samples is collected from the mammal, wherein each biological sample is collected from the mammal at a different time point. A multiplex assay directed to multiple Brucella canis antigens as disclosed above is performed for each collected biological sample. The amounts of antibodies directed to multiple Brucella canis antigens are quantified by measuring the signals (e.g., fluorescence) using a plate reader. The data are represented as median fluorescent intensities (MFI). The progression of Brucella canis infection is monitored by comparing the quantities of fluorescence signals which reflects the amounts of bound antibodies at different time points. Comparison can be done for the amount of each of the antibodies individually over time (e.g., antibodies directed to a B26 antigenic peptide, and antibodies directed to an Omp31 antigenic peptide), or for the combined amounts of the antibodies over time.

In some embodiments, the different time points comprise every 6 hours, every 12 hours, every day, every 2 days, every 3 days, every 4 days, every 5 days, every 6 days, every 7 days, every 8 days, every 9 days, every 10 days, every two weeks or every three weeks. In other embodiments, the different time points comprise every week, every month, every 2 months, every 3 months, every 4 months, every 6 months, and/or every year.

In some embodiments, a method of treating B. canis are provided herein. A method of treatment comprises the described diagnostic method steps, including for example but not limited to employing the described diagnostic methods to determine if a subject is positive for B. canis and treating the subject based on a positive diagnosis. In some embodiments, methods of treating B. canis comprise determining a disease state of the subject (e.g., positive, negative, inconclusive) and treating based on the disease state of the subject. In some embodiments, methods of treating B. canis comprise monitoring a disease state of the subject (e.g., positive, negative, inconclusive) during treatment and continuing/discontinuing treatment based on the disease state of the subject.

When a positive B. canis test is obtained from a dog, it is recommended to confirm with a different test. If the second test comes back negative, an additional test in eight weeks is recommended, maintaining the dog in isolation during this time (Bramlage D J, Fortney W, Kesler R M, Mabray C J, Specialist K, Mason J W, Reinhold H. 2015. Best Practices for Brucella canis Prevention and Control in Dog Breeding Facilities. USDA. Available at: https://www.aphis.usda.gov/animal_welfare/downloads/Brucella_canis_prevention.pdf. Accessed: 03/29/21). When B. canis infection is confirmed, euthanasia is frequently recommended due to the risk to the canine and human population (Cosford K L. 2018. Brucella canis: An update on research and clinical management. Can Vet J 59:74-81). In cases where euthanasia is not elected, such as in a pet dog, spay/neuter is strongly recommended, and treatment with antibiotics may be considered (Greene C E, Carmichael L E. 2012. Canine brucellosis, p. 398-411. In Infectious diseases of the dog and cat, 4th ed.).

In some embodiments, treating comprises treatments known in the art. For example, B. canis infection in dogs can be treated with antibiotics, however complete elimination of the bacteria is often unsuccessful (Cosford K L. 2018. Brucella canis: An update on research and clinical management. Can Vet J 59:74-81). Administering a combination of antibiotics has been shown to improve treatment success. A common combination is a tetracycline, administered orally for 1-2 months, and an aminoglycoside, administered parenterally for 7 days every 2-4 weeks (Cosford K L. 2018. Brucella canis: An update on research and clinical management. Can Vet J 59:74-81; Greene C E, Carmichael L E. 2012. Canine brucellosis, p. 398-411. In Infectious diseases of the dog and cat, 4th ed.); use of three or four antimicrobials in combination, particularly for relapses or when there is ocular/CNS involvement, may further increase treatment efficacy (Greene C E, Carmichael L E. 2012. Canine brucellosis, p. 398-411. In Infectious diseases of the dog and cat, 4th ed.). Monitoring the immune response following treatment can help support success of treatment, however bacteremia may recur within weeks or months, so a successful ‘cure’ should be interpreted with caution, and serologic response should continue to be monitored at 6-9 months intervals following treatment (Greene C E, Carmichael L E. 2012. Canine brucellosis, p. 398-411. In Infectious diseases of the dog and cat, 4th ed.; Hollett R B. 2006. Canine brucellosis: Outbreaks and compliance. Theriogenology 66:575-587).

In some embodiments, a method for treating Brucella canis in a mammal comprises obtaining a sample from a mammal; detecting antibodies reactive to a first antigenic peptide from a BP26 protein of Brucella canis in the sample; detecting antibodies reactive to a second antigenic peptide from an Omp31 protein of Brucella canis in the sample and treating the mammal based on the detection of the first antigenic peptide and/or second antigenic peptide.

The multiplex diagnostic assay disclosed here comprises detecting antibodies reactive to a first antigenic peptide from a BP26 protein of Brucella canis in the sample and/or detecting antibodies reactive to a second antigenic peptide from an Omp31 protein of Brucella canis in the sample and quantifying the amount of antibodies to the first antigenic peptide and/or the amount of antibodies to the second antigenic peptide based on fluorescence signal measurement.

Detection of antibodies reactive to a first antigenic peptide from a BP26 protein of Brucella canis in the sample and/or detection of antibodies reactive to a second antigenic peptide from an Omp31 protein of Brucella canis is indicative of a B. canis infection in the mammal and further comprises an ELISA assay, a fluorescence assay, and/or a colorimetric assay. Treatment of B. canis infection in a mammal comprises treatment with an antibiotic, treatment with more than one antibiotic, treatment with an aminoglycoside, monitoring disease state, and/or quarantining the mammal.

The specific examples listed below are only illustrative and by no means limiting.

### Example 1: Identification of Target Antigens

Probing B. canis Lysate with Serum from Infected Dogs

B. canis lysate (AHDC, Cornell University, Ithaca, NY) was resolved on an SDS-PAGE gel and transferred to a PVDF membrane for subsequent immunoblotting. Serum from dogs that were previously positive or negative by the reference assay (AHDC, Cornell University, Ithaca, NY) were used to probe the immunoblot, diluted at 1:400, and reactive bands were detected with anti-canine immunoglobulin (Jackson Immunoresearch Laboratories, West Grove, PA), diluted 1:20,000. Infection-specific immunoreactive bands were identified at ˜30 kDa, ˜25 kDa and ˜9 KDa. Subsequently, bands corresponding to ˜30 kDa, 25 kDa and ˜9 kDa were excised from a gel and analyzed by mass spectrometry for identification. The top identities from each band, ABCt (WP_004687190.1), BP26 (YP_001593309.1) and HRL18 (NC_010103.1, c1079819-1079544), respectively, were cloned from B. canis genomic DNA. Of these proteins, only BP26 (SEQ ID NO: 1) was selected for use in the current disclosure.

Scanning Protein Databank for Seroreactive Antigens

To identify additional potential antigen candidates, a search of the protein databank was performed. This search yielded an additional four potential candidates: TRAP (AOG40818.1), Omp25 (ENX70214.1), mocB (WP_004690331), and Omp31² (ERU01676.1). Each additional candidate was cloned from B. canis genomic DNA. Of these proteins, full-length recombinant Omp31 protein was the most promising candidate, but the purity of the purified recombinant protein was difficult to establish.

### Example 2: Identification and Production of Immunoreactive Region of Omp31

To produce a pure antigenic target from Omp31, a transmembrane topology prediction was performed. First, the signal sequence was removed, as defined by SignalP-4.1 (http://www.cbs.dtu.dk/services/SignalP-4.1/) prediction. Next, the sequence was run through the TMpred software. Based on analysis of the mean burial propensity, a region at the c-terminal domain of the protein, representing amino acids 48-77 of the Omp31 protein, was hypothesized to be an externally exposed loop that would have increased exposure for immune recognition. This Omp31 peptide (Omp31pep, SEQ ID NO: 8) was synthesized by a laboratory with expertise in peptide synthesis and conjugated to a carrier protein, specifically bovine serum albumin (BSA), through a Cysteine amino acid added to the C-terminus of the peptide (Biosynthesis, Lewisville, TX).

### Example 3: Further Optimization of Omp31 Peptide

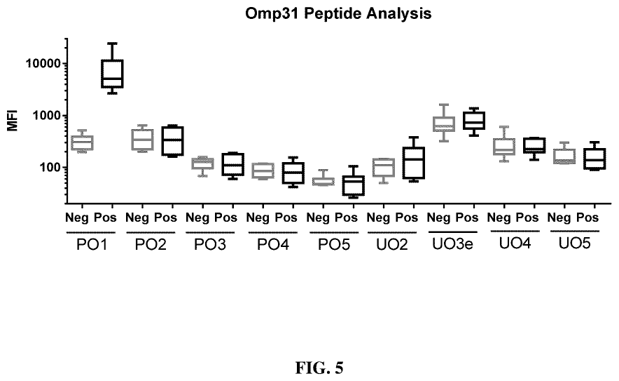

A reference assay as shown in FIG. 5 was performed to identify a smaller 17aa acid peptide (PO1, SEQ ID NO: 9) within the Omp31pep sequence that further improved the sensitivity and specificity of the assay. The peptide was synthesized by a laboratory with expertise in peptide synthesis (LifeTein, Hiltsborough, NJ) and conjugated, through a Cysteine amino acid residue added to the C-terminus of the peptide, to a maleimide-activated BSA carrier protein (Pierce Biotechnology, Rockford, IL).

### Example 4: Cloning of BP26 Protein

The BP26 was initially cloned from the genome of B. canis using BP26F (SEQ ID NO: 3): 5′cgctcATGAACACTCGTGCTAGCAATTTTCTCG and BP26R (SEQ ID NO: 4): 5′gcgggatccCTTGATTTCAAAAACGACATTGACCGATACGTT. This construct was digested with BpsHI and BamHI and cloned in to a pQE-60 vector (Quigen Inc., Germantown, MD) digested with NcoI and BamHI, leading to the addition of a c-terminal 6× Histidine tag. The protein was subsequently modified by removal of the N-terminal transmembrane domain in order to improve expression efficiency. This subcloning was performed using primers BP26Forward: 5′cgcccatggCACAGGAGAATCAGATGACG (SEQ ID NO: 5) and BP26Reverse: 5′cgcagatctCTTGATTTCAAAAACGACATTGAC (SEQ ID NO: 6), digested by NcoI and BglII and inserted back into the same pQE60 plasmid to produce pQE60_tBP26.

### Example 5: Expression and Purification of the BP26 Protein

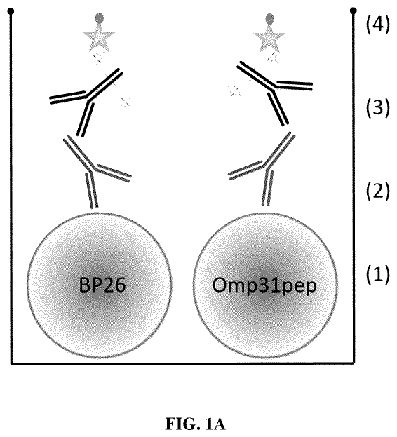

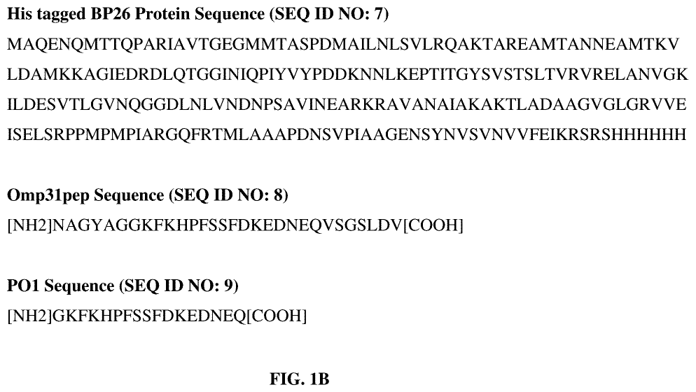

The BP26 protein was expressed in the SG13009 E. coli expression host (Quigen Inc., Germantown, MD) transformed with pQE60_tBP26, grown in LB broth containing kanamycin and ampicillin. Protein expression was induced by the addition of 1 mM IPTG. The bacteria were suspended in urea lysis buffer (100 mM sodium phosphate, 10 mM Tris, 8M urea, pH8.0) and subjected to sonication. The lysate was then cleared by centrifugation, mixed 1:5 with 40 mM imidazole buffer (40 mM imidazole, 20 mM sodium phosphate, 0.5M NaCl), and filter sterilized. The His-tagged protein as shown in FIG. 1B, was purified using a HisTrapFF Ni-NTA column (GE Healthcare, Piscataway, NJ). Eluted protein was dialyzed into phosphate buffered saline, pH 7.4. Protein concentrations were determined by BCA assay (Pierce, Rockford, IL).

### Example 6: Coupling of Antigens to Fluorescent Beads

A volume containing 200 μg of each B. canis antigen was coupled to 10⁷ fluorescent beads (Luminex Corp). BP26 was coupled to bead 33 and Omp31pep was coupled to bead 38. Additional antigens tested early in assay development, including HRL18, ABCt, TRAP, Omp25, mocB, and Omp31 were coupled to beads 37, 34, 35, 36, 37, and 38 respectively; the recombinant Omp31 protein was abandoned in favor of Omp31pep. The coupling was performed in accordance with manufacturer recommendations. Briefly, all steps were conducted at room temperature, beads were resuspended by vortex and sonication, incubations were in the dark, and beads were pelleted by centrifugation at 4700×g for 4 minutes. 800 μl of bead stock was washed with water. The beads were activated by first suspending in 160 μl of 100 mM sodium phosphate pH 6.2. Next, 20 μl of 50 mg/mL Sulfo-NHS (Pierce Biotechnology Inc., Rockford, IL), followed by 20 μl of 50 mg/mL EDC (Pierce Biotechnology Inc., Rockford, IL) were added and incubated for 20 minutes. The beads were then washed twice with 500 μl of 50 mM MES pH 5.0 (Sigma-Aldrich Inc., St. Louis, MO). The activated beads were mixed with the MES buffer and the appropriate protein in a 1 mL volume and rotated for 3 hr to complete the coupling. Next, the beads were incubated in 1 mL PBN for 30 min, and finally the beads were washed three times in 2 mL of PBN with 0.02% Tween 20. Beads were then counted and stored in the dark at 2-8° C.

### Example 7: Fluorescent Bead-Based Assay

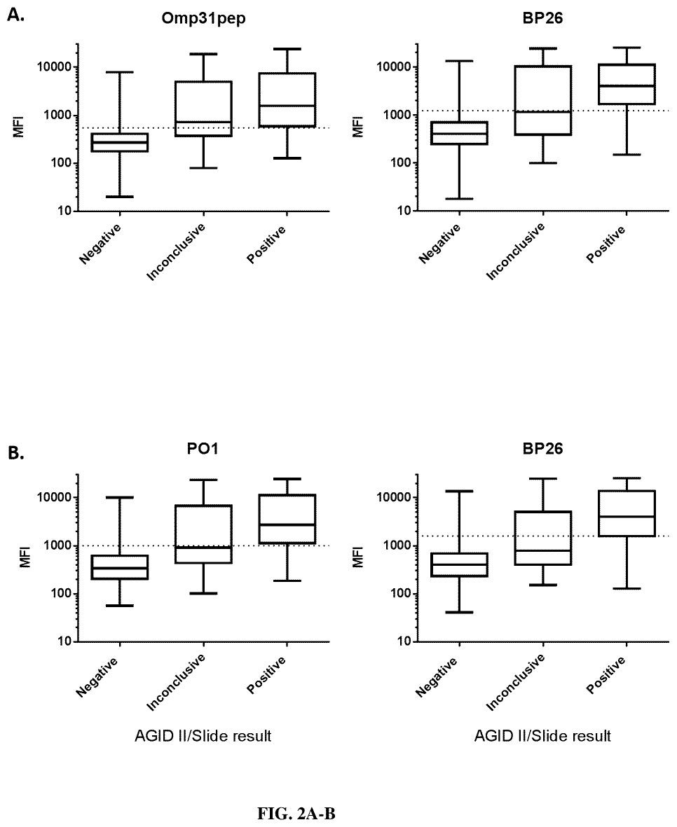

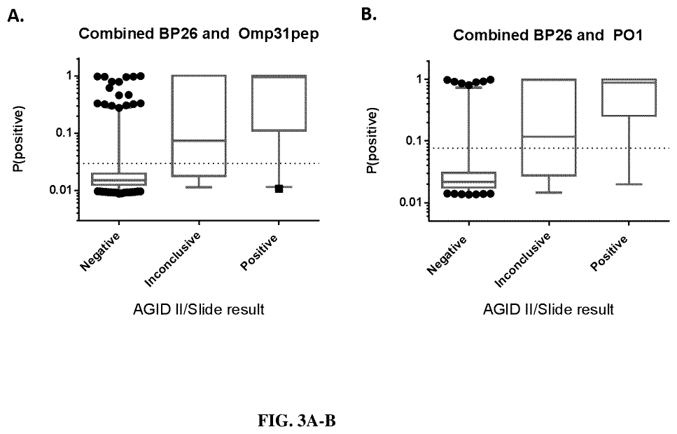

An illustration of the multiplex assay utilized for this disclosure is shown in FIG. 1A. Beads 33 and 38 coupled with BP26 and Omp31pep, respectively, were sonicated, mixed, and diluted in a blocking buffer, phosphate buffered saline with bovine serum albumin and sodium azide (PBN), to a final concentration of 10⁵ beads/mL. Canine serum sample were diluted 1:600 in PBN. Previously tested negative, low positive and high positive canine sera were set on each assay plate as positive and negative controls. Millipore Multiscreen HTS plates (Millipore, Danvers, MA) were wetted for 2 minutes with phosphate buffered saline containing 0.05% Tween 20 (PBST); an ELx50 plate washer (Biotek Instruments Inc., Winooski, VT) was used for adding and aspirating PBST for this incubation and subsequent wash steps. After aspirating PBST, 50 μl of each diluted serum and control sample was added to the appropriate wells of the plate. Next, 50 μl of the bead solution was added to each well, and the plate was incubated for 30 minutes, shaking, at room temperature. After washing the serum-incubated beads three times, 50 μl of biotinylated rabbit anti-dog IgG(H+L) (Jackson Immunoresearch Laboratories, West Grove, PA), diluted 1:5000 in PBN, was added to each well and incubated for 30 minutes as above. Following a wash step, 50 μl of streptavidin-phycoerythrin (Invitrogen, Carlsbad, CA), diluted 1:100 in PBN, was added to each well. Plates were incubated for 30 minutes as above and then washed. Beads were resuspended in 1001 of PBN and incubated for 15 minutes as above. The resuspended beads were then analyzed in a Luminex IS 100 instrument ((Luminex Corp.) using BioPlex software (Bio-Rad Laboratories Inc., Hercules, CA). The data were reported as median fluorescent intensities (MFI) as shown in FIG. 2 and FIG. 3.

The following tables represent the interpretation of the multiplex assay data.

TABLE 1
Result interpretation. Cut-off values were determine based on ROC curve analysis, for each
antigen independently. (A) Interpretation table. (B., C.) Distribution of results described
herein. These results make it clear that a subset of animals respond to only one of the
two antigens. Utilizing both results in interpretation allows for higher sensitivity.

B. BP26 (MFI)

| A. BP26 result | | | | | | <1200 | ≥1200, <3600 | ≥3600 | total |
|---|---|---|---|---|---|---|---|---|---|
| Omp31 | Negative | Equivocal | Positive | Omp31pep | <600 | 1490 | 126 | 36 | 1652 |
| peptide | Equivocal | Suspect | Positive | (MFI) | ≥600, <1800 | 148 | 47 | 31 | 226 |
| result | Positive | Positive | Positive | | ≥1800 | 18 | 25 | 53 | 96 |
| | | | | | total | 1656 | 198 | 120 | 1974 |

| | C. BP26 (MFI) | | | | |
|---|---|---|---|---|---|
| | | <1200 | ≥1200, <3600 | ≥3600 | total |
| PO1 | <1000 | 612 | 74 | 26 | 712 |
| (MFI) | ≥1000, <2000 | 48 | 15 | 17 | 80 |
| | ≥2000 | 30 | 16 | 30 | 76 |
| | total | 690 | 105 | 73 | 868 |

TABLE 2
Comparison to the reference assay. Interpretation of the
B. canis Multiplex Assay results in comparison with the
AGID/2ME-RSAT results described herein with either
A. Omp31pep or B. PO1 as the second antigen in the assay.
Although the B. canis Multiplex Assay qualifies more
samples in the ‘equivocal’ range, the quantitative nature of
the assay allows for easier comparison in follow-up testing.
A. Reference Results
B. canis Multiplex Assay (BP26, Omp31pep) Results

| | | Positive | Suspect | Equivocal | Negative |
|---|---|---|---|---|---|
| | | 163 | 47 | 274 | 1490 |
| Positive | 130 | 96 | 8 | 16 | 10 |
| Inconclusive | 60 | 28 | 2 | 11 | 19 |
| Negative | 1784 | 39 | 37 | 247 | 1461 |

B. Reference Results
B. canis Multiplex Assay (BP26, PO1) Results

| | | Positive | Suspect | Equivocal | Negative |
|---|---|---|---|---|---|
| | | 119 | 15 | 122 | 612 |
| Positive | 88 | 69 | 7 | 9 | 3 |
| Inconclusive | 33 | 17 | 1 | 5 | 10 |
| Negative | 747 | 33 | 7 | 108 | 599 |

TABLE 3
Sensitivity and Specificity at the lower and upper cut-off values ^{a}.

| | | Cut-off | | | | |
|---|---|---|---|---|---|---|
| | Assay | value^{d} | Sensitivity | (95% CI) | Specificity | (95% CI) |
| Inconclusives | Reference Assay | | 0.673 | (0.554- | 0.992 | (0.981- |
| Negative ^{b} | | | | 0.803) | | 0.999) |
| | PO1 | 1000 MFI | 0.837 | (0.761- | 0.883 | (0.856- |
| | | | | 0.901) | | 0.910) |
| | | 2000 MFI | 0.633 | (0.548- | 0.964 | (0.946- |
| | | | | 0.718) | | 0.980) |
| | Reference Assay | | 0.797 | (0.671- | 0.996 | (0.985- |
| | | | | 0.949) | | 0.999) |
| | BP26 | 2400 MFI | 0.691 | (0.611- | 0.936 | (0.917- |
| | | | | 0.775) | | 0.955) |
| | | 3600 MFI | 0.588 | (0.506- | 0.965 | (0.947- |
| | | | | 0.668) | | 0.978) |
| | Multiplex ^{c} | lower | 0.950 | | 0.826 | |
| | | values | | | | |
| | | upper | 0.849 | | 0.930 | |
| | | values | | | | |
| Inconclusives | Reference Assay | | 0.961 | (0.862- | 0.988 | (0.972- |
| Positive ^{b} | | | | 0.999) | | 0.999) |
| | PO1 | 1000 MFI | 0.792 | (0.722- | 0.861 | (0.839- |
| | | | | 0.859) | | 0.882) |
| | | 2000 MFI | 0.624 | (0.546- | 0.961 | (0.947- |
| | | | | .0699) | | 0.975) |
| | Reference Assay | | 0.962 | (0.881- | 0.983 | (0.965- |
| | | | | 0.999) | | 0.999) |
| | BP26 | 2400 MFI | 0.582 | (0.505- | 0.966 | (0.954- |
| | | | | 0.659) | | 0.978) |
| | | 3600 MFI | 0.524 | (0.446- | 0.978 | (0.967- |
| | | | | 0.598) | | 0.987) |
| | Multiplex ^{c} | lower | 0.913 | | 0.832 | |
| | | values | | | | |
| | | upper | 0.821 | | 0.939 | |
| | | values | | | | |

^{a} The non-parametric estimation of ROC curves without gold standard test was run with a previously described R-package (1) and included three groups: export samples (n = 71), samples from a set of outbreak cases (n = 250), and other diagnostic submissions (n = 1192). This analysis assumes the reference test and the antigen detection assays that make-up the B. canis Multiples Assay are independent, conditional on disease status.
^{b} Sensitivity and specificity for both PO1 and BP26 are determined in comparison to the reference assay, with reference assay samples results of ‘inconclusive’ considered either ‘positive’ or ‘negative’.
^{c} A combined sensitivity and specificity for the multiplex assay is calculated as parallel tests, where either test being positive is considered as evidence of disease presence.
^{d}Upper and lower cut-off values for each antigen in the Multiplex assay were determined based on ROC curve analysis. A sample with a value above the upper cut-off for either antigen would be considered ‘positive’; a sample below the cut-off value for both antigens would be considered ‘negative’; all other samples would be considered ‘equivocal’. (Wang C, Turnbull BW, Gröhn YT, Nielsen SS. 2007. Nonparametric estimation of ROC curves based on Bayesian models when the true disease state is unknown. JABES 12: 128-146.)

Table 4 as shown below, summarizes the protein/peptide and primer sequences used in the disclosure and the corresponding description.

| Name | Description |
|---|---|
| BP26 (SEQ ID NO: 1) | Full length BP26 protein (GenBank ID: |
| | AIJ81780.1) from Brucella canis. |
| Omp31 (SEQ ID NO: 2) | Full length Omp31 protein (GenBank ID: |
| | AAL27296.1) from Brucella canis. |
| BP26F (SEQ ID NO: 3) | Primer sequence from Brucella canis |
| | genome. |
| BP26R (SEQ ID NO: 4) | Primer sequence from Brucella canis |
| | genome. |
| BP26Forward (SEQ ID NO: | Primer sequence from Brucella canis |
| 5) | genome. |
| BP26Reverse (SEQ ID NO: | Primer sequence from Brucella canis |
| 6) | genome. |
| BP26 His tagged (SEQ ID | His tagged BP26 Protein Sequence. |
| NO: 7) | |
| Omp31pep (SEQ ID NO: 8) | Fragment of Omp31 protein Sequence. |
| PO1 (SEQ ID NO: 9) | Fragment of Omp31pep Sequence. |
| PO2 (SEQ ID NO: 10) | Fragment of Omp31pep Sequence. |
| PO3 (SEQ ID NO: 11) | Fragment of Omp31pep Sequence. |
| PO4 (SEQ ID NO: 12) | Fragment of Omp31pep Sequence. |
| PO5 (SEQ ID NO: 13) | Fragment of Omp31pep Sequence. |
| UO2 (SEQ ID NO: 14) | Fragment of Omp31pep Sequence. |
| UO3 (SEQ ID NO: 15) | Fragment of Omp31pep Sequence. |
| UO4 (SEQ ID NO: 16) | Fragment of Omp31pep Sequence. |
| UO5 (SEQ ID NO: 17) | Fragment of Omp31pep Sequence. |

What is claimed is:

1. A kit for detecting immune responses to a Brucella canis infection in a mammal comprising:
a multiplex capture reagent comprising a first antigenic peptide from a Brucella canis BP26 protein linked to a first detectable label, and a second antigenic peptide from a Brucella canis Omp31 protein linked to a second detectable label;
a detection antibody against antibodies of the mammal, wherein the detection antibody is linked to a third detectable label, and wherein the first detectable label, the second detectable label and the third detectable labels are different from each other;
and optionally a solid substrate and instructions for detecting the presence of antibodies to Brucella canis.

2. The kit of claim 1, wherein the BP26 protein comprises the amino acid sequence of SEQ ID NO: 1 or an amino acid sequence at least 95% identical to SEQ ID NO: 1.

3. The kit of claim 1 or 2, wherein the first antigenic peptide comprises at least 10, at least 20, at least 30, at least 40, at least 50, at least 60, at least 70, at least 80, at least 90, at least 100 amino acids, at least 120 amino acids, at least 150 amino acids, at least 180 amino acids, at least 200 amino acids, at least 220 amino acids or all of the BP26 protein.

4. The kit according to any one of claims 1-3, wherein the first antigenic peptide comprises a fragment of the BP26 protein without the N-terminal transmembrane domain of the BP26 protein.

5. The kit according to any one of claims 1-4, wherein the Omp31 protein comprises the amino acid sequence of SEQ ID NO: 2 or an amino acid sequence at least 95% identical to SEQ ID NO: 2.

6. The kit according to any one of claims 1-5, wherein the second antigenic peptide comprises at least 10 amino acids of the Omp31 protein.

7. The kit according to any one of claims 1-5, wherein the second antigenic peptide comprises an extracellular region of the Omp31 protein.

8. The kit of claim 7, wherein the extracellular region of the Omp31 protein comprises an amino acid sequence that is at least 95% identical to SEQ ID NO: 8.

9. The kit of claim 7, wherein the extracellular region of the Omp31 protein comprises an amino acid sequence as shown in SEQ ID NO: 8 or 9.

10. The kit according to any one of claims 1-9, wherein the first detectable label is a first fluorescent bead and the second detectable label is a second fluorescent bead.

11. The kit according to any one of claims 1-10, wherein the first detectable label, the second detectable label and the third detectable label provide fluorescent signals.

12. The kit according to any one of claims 1-11, wherein the mammal is a dog or a human.

13. The kit according to any one of claims 1-12, wherein the solid substrate is selected from a multi-well plate or a laminar flow device.

14. The kit according to any one of claims 1-13, wherein the capture reagent comprises a third antigenic peptide from a Brucella canis protein.

15. A method for simultaneous detection of antibodies directed to multiple Brucella canis antigens in a mammal comprising:
(a) providing a capture reagent comprising a first antigenic peptide from a BP26 protein of Brucella canis linked to a first detectable label, and a second antigenic peptide from an Omp31 protein of Brucella canis linked to a second detectable label, wherein the first detectable label and the second detectable label are different from each other;
(b) contacting the capture reagent with a biological sample from the mammal to allow for antibodies present in the sample to bind to the antigenic peptides in the capture reagent, thereby forming a first complex between the first antigenic peptide and an antibody, and a second complex between the second antigenic peptide and an antibody;
(c) contacting the capture reagent after step (b) with a detection antibody, wherein the detection antibody is linked to a third detectable label that is different from the first detectable label and the second detectable label, to allow for the detection antibody to bind to the antibodies in the first and second complexes; and
(d) detecting the detection antibody bound to the antibody in first complex and detecting detection antibody bound to the antibody in the second complex.

16. The method of claim 15, further comprising quantifying the amount of detection antibody bound to the antibody in the first complex and the amount of detection antibody bound to the antibody in the second complex.

17. The method of claim 15 or 16, wherein the BP26 protein comprises the amino acid sequence of SEQ ID NO: 1.

18. The method according to any one of claims 15-17, wherein the first antigenic peptide comprises at least 10, at least 20, at least 30, at least 40, at least 50, at least 60, at least 70, at least 80, at least 90, at least 100 amino acids, at least 120 amino acids, at least 150 amino acids, at least 180 amino acids, at least 200 amino acids, at least 220 amino acids or all of the BP26 protein.

19. The method according to any one of claims 15-18, wherein the first antigenic peptide comprises a fragment of the BP26 protein without the N-terminal transmembrane domain of the BP26 protein.

20. The method according to any one of claims 15-19, wherein the Omp31 protein comprises the amino acid sequence of SEQ ID NO: 2.

21. The method according to any one of claims 15-20, wherein the second antigenic peptide comprises at least 10 amino acids of the Omp31 protein.

22. The method according to any one of claims 15-21, wherein the second antigenic peptide comprises an extracellular region of the Omp31 protein.

23. The method of claim 22, wherein the extracellular region of the Omp31 protein comprises an amino acid sequence that is at least 95% identical to SEQ ID NO:8.

24. The method of claim 22, wherein the extracellular region of the Omp31 protein comprises an amino acid sequence as shown in SEQ ID NO: 8 or 9.

25. The method according to any one of claims 15-24, wherein the first detectable label is a first fluorescent bead and the second detectable label is a second fluorescent bead.

26. The method according to any one of claims 15-24, wherein the first detectable label, the second detectable label and the third detectable label are fluorescent labels.

27. The method of claim 25 or claim 26, wherein the detection is achieved by a flow cytometer or a plate reader.

28. The method according to any one of claims 15-27, wherein the mammal is a dog or a human.

29. The method according to any one of claims 15-28, wherein the biological sample is selected from blood, plasma, serum, saliva, urine, synovial and cerebrospinal fluid, milk and tissue homogenates.

30. The method according to any one of claims 15-29, wherein a combination of a positive detection of the detection antibody bound to the antibody in first complex and a positive detection of the detection antibody bound to the antibody in the second complex is indicative of a Brucella canis infection.

31. A method for monitoring progression of a Brucella canis infection in a mammal comprising:
(a) providing a plurality of biological samples collected from the mammal, wherein each biological sample is collected from the mammal at a different time point;
(b) measuring the amount of antibodies directed to multiple Brucella canis antigens in each biological sample,
wherein each measuring comprises:
(i) providing a biological sample from one time point;
(ii) providing a capture reagent comprising a first antigenic peptide from a BP26 protein of Brucella canis linked to a first detectable label, and a second antigenic peptide from an Omp31 protein of Brucella canis linked to a second detectable label, wherein the first detectable label and the second detectable label are different from each other;
(iii) contacting the capture reagent with the biological sample to allow for antibodies present in the sample to bind to the antigenic peptides in the capture reagent, thereby forming a first complex between the first antigenic peptide and an antibody, and a second complex between the second antigenic peptide and an antibody;
(iv) contacting the capture reagent after step (iii) with a detection antibody, wherein the detection antibody is linked to a third detectable label that is different from the first detectable label and the second detectable label, to allow for the detection antibody to bind to the antibodies in the first and second complexes, and
(v) quantifying the amount of detection antibody bound to the antibody in the first complex and the amount of detection antibody bound to the antibody in the second complex;
and
(c) comparing the amounts measured in step (b) for the different time points, thereby monitoring the progression of Brucella canis infection.

32. The method of claim 31, wherein the different time points comprise every 6 hours, every 12 hours, every day, every 2 days, every 3 days, every 4 days, every 5 days, every 6 days, every 7 days, every 8 days, every 9 days, every 10 days, every two weeks or every three weeks.

33. The method of claim 31, wherein the different time points comprise every week, every month, every 2 months, every 3 months, every 4 months, every 6 months, and/or every year.

34. The method according to any one of claims 31-33, wherein the BP26 protein comprises the amino acid sequence of SEQ ID NO: 1 or an amino acid sequence at least 95% identical to SEQ ID NO: 1.

35. The method according to any one of claims 31-34, wherein the first antigenic peptide comprises at least 10, at least 20, at least 30, at least 40, at least 50, at least 60, at least 70, at least 80, at least 90, at least 100 amino acids, at least 120 amino acids, at least 150 amino acids, at least 180 amino acids, at least 200 amino acids, at least 220 amino acids or all of the BP26 protein.

36. The method according to any one of claims 31-35, wherein the first antigenic peptide comprises a fragment of the BP26 protein without the N-terminal transmembrane domain of the BP26 protein.

37. The method according to any one of claims 31-36, wherein the Omp31 protein comprises the amino acid sequence of SEQ ID NO: 2 or an amino acid sequence at least 95% identical to SEQ ID NO: 2.

38. The method according to any one of claims 31-37, wherein the second antigenic peptide comprises at least 10 amino acids of the Omp31 protein.

39. The method according to any one of claims 31-38, wherein the second antigenic peptide comprises an extracellular region of the Omp31 protein.

40. The method of claim 39, wherein the extracellular region of the Omp31 protein comprises an amino acid sequence that is at least 95% identical to SEQ ID NO: 8.

41. The method of claim 39, wherein the extracellular region of the Omp31 protein comprises an amino acid sequence as shown in SEQ ID NO: 8 or 9.

42. The method according to any one of claims 31-41, wherein the first detectable label is a first fluorescent bead and the second detectable label is a second fluorescent bead.

43. The method according to any one of claims 31-42, wherein the first detectable label, the second detectable label and the third detectable label are fluorescent labels.

44. The method of claim 42 or claim 43, wherein the detection is achieved by a flow cytometer or a plate reader.

45. The method according to any one of claims 31-44, wherein the mammal is a dog or a human.

46. The method according to any one of claims 31-45, wherein the biological sample is selected from blood, plasma, serum, saliva, urine, synovial and cerebrospinal fluid, milk and tissue homogenates.

47. A method for treating Brucella canis in a mammal comprising:
(a) obtaining a sample from a mammal;
(b) detecting antibodies reactive to a first antigenic peptide from a BP26 protein of Brucella canis in the sample;
(c) detecting antibodies reactive to a second antigenic peptide from an Omp31 protein of Brucella canis in the sample; and
(d) treating the mammal based on a detection of antibodies to the first antigenic peptide and/or a detection of antibodies to the second antigenic peptide.

48. The method of claim 47, further comprising quantifying an amount of antibodies to the first antigenic peptide and/or quantifying an amount of antibodies to the second antigenic peptide.

49. The method of claim 47 or 48, wherein the BP26 protein comprises the amino acid sequence of SEQ ID NO: 1 or an amino acid sequence at least 95% identical to SEQ ID NO.

50. The method according to any one of claims 47-49, wherein the first antigenic peptide comprises at least 10, at least 20, at least 30, at least 40, at least 50, at least 60, at least 70, at least 80, at least 90, at least 100 amino acids, at least 120 amino acids, at least 150 amino acids, at least 180 amino acids, at least 200 amino acids, at least 220 amino acids or all of the BP26 protein.

51. The method according to any one of claims 47-50, wherein the first antigenic peptide comprises a fragment of the BP26 protein without the N-terminal transmembrane domain of the BP26 protein.

52. The method according to any one of claims 47-51, wherein the Omp31 protein comprises the amino acid sequence of SEQ ID NO: 2 or an amino acid sequence at least 95% identical to SEQ ID NO: 2.

53. The method according to any one of claims 47-52, wherein the second antigenic peptide comprises at least 10 amino acids of the Omp31 protein.

54. The method according to any one of claims 47-53, wherein the second antigenic peptide comprises an extracellular region of the Omp31 protein.

55. The method of claim 54, wherein the extracellular region of the Omp31 protein comprises an amino acid sequence that is at least 95% identical to SEQ ID NO: 8.

56. The method of claim 54, wherein the extracellular region of the Omp31 protein comprises an amino acid sequence as shown in SEQ ID NO: 8 or 9.

57. The method of claim 47, wherein detecting antibodies reactive to a first antigenic peptide from a BP26 protein of Brucella canis in the sample and/or detecting antibodies reactive to a second antigenic peptide from an Omp31 protein of Brucella canis in the sample comprises an ELISA assay, a fluorescence assay, and/or a colorimetric assay.

58. The method of any one of claims 47-57, wherein the mammal is a dog or a human.

59. The method of any one of claims 47-58, wherein the biological sample is selected from blood, plasma, serum, saliva, urine, synovial and cerebrospinal fluid, milk and tissue homogenates.

60. The method of claim any one of claims 47-59, wherein a combination of a positive detection of antibodies reactive to a first antigenic peptide from a BP26 protein of Brucella canis in the sample and a positive detection of antibodies reactive to a second antigenic peptide from an Omp31 protein of Brucella canis is indicative of a B. canis infection in the mammal.

61. The method of any one of claims 47-60, wherein treating comprises treating with an antibiotic, treating with more than one antibiotic, treating with an aminoglycoside, monitoring disease state, and/or quarantining the mammal.


SEQUENCE LISTING (17 sequences)

SEQ ID NO: 1 - protein, 250 aa, Brucella canis
MNTRASNFLAASFSTIMLVGAFSLPAFAQENQMTTQPARIAVTGEGMMTASPDMAILNLSVLRQAKTAREAMTANNEAMTKVLDAMKKAGIEDRDLQTGGINIQPIYVYPDDKNNLKEPTITGYSVSTSLTVRVRELANVGKILDESVTLGVNQGGDLNLVNDNPSAVINEARKRAVANAIAKAKTLADAAGVGLGRVVEISELSRPPMPMPIARGQFRTMLAAAPDNSVPIAAGENSYNVSVNVVFEIK

SEQ ID NO: 2 - protein, 240 aa, Brucella canis
MKSVILASIAAMFATSAMAADVVVSEPSAPTAAPVDTFSWTGGYIGINAGYAGGKFKHPFSSFDKEDNEQVSGSLDVTAGGFVGGVQAGYNWQLDNGVVLGAETDFQGSSVTGPISAGASGLEGKAETKVEWFGTVRARLGYTATERLMVYGTGGLAYGKVKSAFNLGDDASALHTWSDKTKAGWTLGAGAEYAINNNWTLKSEYLYTDLGKRNLVDVDNSFLESKVNFHTVRVGLNYKF

SEQ ID NO: 3 - DNA, 33 bp, Brucella canis
cgctcatgaacactcgtgctagcaattttctcg

SEQ ID NO: 4 - DNA, 42 bp, Brucella canis
gcgggatcccttgatttcaaaaacgacattgaccgatacgtt

SEQ ID NO: 5 - DNA, 29 bp, Brucella canis
cgcccatggcacaggagaatcagatgacg

SEQ ID NO: 6 - DNA, 33 bp, Brucella canis
cgcagatctcttgatttcaaaaacgacattgac

SEQ ID NO: 7 - protein, 234 aa, Artificial Sequence
  His tagged BP26
MAQENQMTTQPARIAVTGEGMMTASPDMAILNLSVLRQAKTAREAMTANNEAMTKVLDAMKKAGIEDRDLQTGGINIQPIYVYPDDKNNLKEPTITGYSVSTSLTVRVRELANVGKILDESVTLGVNQGGDLNLVNDNPSAVINEARKRAVANAIAKAKTLADAAGVGLGRVVEISELSRPPMPMPIARGQFRTMLAAAPDNSVPIAAGENSYNVSVNVVFEIKRSRSHHHHHH

SEQ ID NO: 8 - protein, 30 aa, Artificial Sequence
  Omp31pep (Fragment of Omp31 protein)
NAGYAGGKFKHPFSSFDKEDNEQVSGSLDV

SEQ ID NO: 9 - protein, 17 aa, Artificial Sequence
  PO1 (Fragment of Omp31pep)
GKFKHPFSSFDKEDNEQ

SEQ ID NO: 10 - protein, 15 aa, Artificial Sequence
  PO2 (Fragment of Omp31pep)
NAGYAGGKFKHPFSS

SEQ ID NO: 11 - protein, 15 aa, Artificial Sequence
  PO3 (Fragment of Omp31pep)
FDKEDNEQVSGSLDV

SEQ ID NO: 12 - protein, 15 aa, Artificial Sequence
  PO4 (Fragment of Omp31pep)
GGYIGINAGYAGGKF

SEQ ID NO: 13 - protein, 20 aa, Artificial Sequence
  PO5 (Fragment of Omp31pep)
EQVSGSLDVTAGGFVGGVQA

SEQ ID NO: 14 - protein, 20 aa, Artificial Sequence
  UO2 (Fragment of Omp31 protein)
MAADVVVSEPSAPTAAPVDT

SEQ ID NO: 15 - protein, 24 aa, Artificial Sequence
  UO3 (Fragment of Omp31 protein)
YNWQLDNGVVLGAETDFQGSSVTG

SEQ ID NO: 16 - protein, 20 aa, Artificial Sequence
  UO4 (Fragment of Omp31 protein)
MVYGTGGLAYGKVKSAFNLG

SEQ ID NO: 17 - protein, 20 aa, Artificial Sequence
  UO5 (Fragment of Omp31 protein)
NSFLESKVNFHTVRVGLNYK